(19) United States
(12) Patent Application Publication
(10) Pub. No.: US 2009/0205988 A1
(43) Pub. Date: Aug. 20, 2009

(54) ERGONOMIC DISPOSABLE TRAY FOR HOLDING STERILE SURGICAL COMPONENTS

(75) Inventors: Craig Edwards, Mission Viejo, CA (US); John I. Muri, Laguna Niguel, CA (US)

Correspondence Address: ABBOTT MEDICAL OPTICS, INC., 1700 E. ST. ANDREW PLACE, SANTA ANA, CA, 92705, US

(73) Assignee: Advanced Medical Optics, Inc., Santa Ana, CA (US)

(21) Appl. No.: 12/032,569

(22) Filed: Feb. 15, 2008

(51) Int. Cl. A61B 19/02; B65D 1/34

(52) U.S. Cl. 206/370; 206/557

(57) ABSTRACT

The field of the invention relates to medical packaging and, more particularly, to trays for holding sterile surgical components. In one embodiment, an ergonomic disposable tray is provided which can rest easily in the palm of the hand and be easily grasped. Moreover, the tray includes the addition of a raised triangular protrusion in all four opposing corners, to make opening the tray equally efficient for both right and left handed users. These features will allow the user to maintain control of the tray while peeling back the lid, keeping the internal components sterile.

### FIELD OF THE INVENTION

The field of the invention relates to medical packaging and, more particularly, to trays for holding sterile surgical components.

### BACKGROUND OF THE INVENTION

Disposable trays are employed to package, or contain, the sterile components required for various surgeries, e.g., cataract surgery. In order to accommodate the number of components required for surgery, the tray can be large in size and weigh as much as one pound each. The top surface of the tray is covered with a sterile lid which is heat sealed in place. The lid forms an airtight seal with the tray to keep the contents of the tray sterile until the lid is removed. Due to the sterile environment required for surgery, the tray must be handled correctly so the internal components are not damaged or contaminated.

In order to prepare for surgery, a nurse must carefully balance the tray in one hand while simultaneously opening the lid. Specifically, the nurse is required to peel back the lid by starting at one of two corners of the tray. Since the lid is heat sealed into place, the force needed to peel back the lid occasionally can lift the tray out of the nurse's hand and spill the sterile contents onto the floor, where they become contaminated. This event disrupts the surgical environment and creates extra cost for the surgeon and patient as the contaminated pack must be replaced.

### SUMMARY OF THE INVENTION

The field of the invention relates to medical packaging and, more particularly, to trays for holding sterile surgical components. In one embodiment, an ergonomic disposable tray is provided which can rest easily in the palm of the hand and be easily grasped. Moreover, the tray includes the addition of a raised triangular protrusion in all four opposing corners, to make opening the tray equally efficient for both right and left handed users. These features will allow the user to maintain control of the tray while peeling back the lid, keeping the internal components sterile.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The present invention will become more fully understood from the detailed description given and the accompanying drawings which are given by way of illustration only, and thus are not limitative of the present invention, and wherein:

FIG. 1 is an isometric view of a surgical tray embodying the principles of the present invention.

FIG. 2 is a top view of the surgical tray shown in FIG. 1;

FIG. 3 is the front view of the surgical tray shown in FIG. 1;

FIG. 4 is the side view of the surgical tray shown in FIG. 1;

FIG. 5 is a top view of the flange as shown in FIG. 1;

FIG. 6 is a top view of section one of the flange in FIG. 4.

### DETAILED DESCRIPTION OF THE PREFERRED EMBODIMENTS

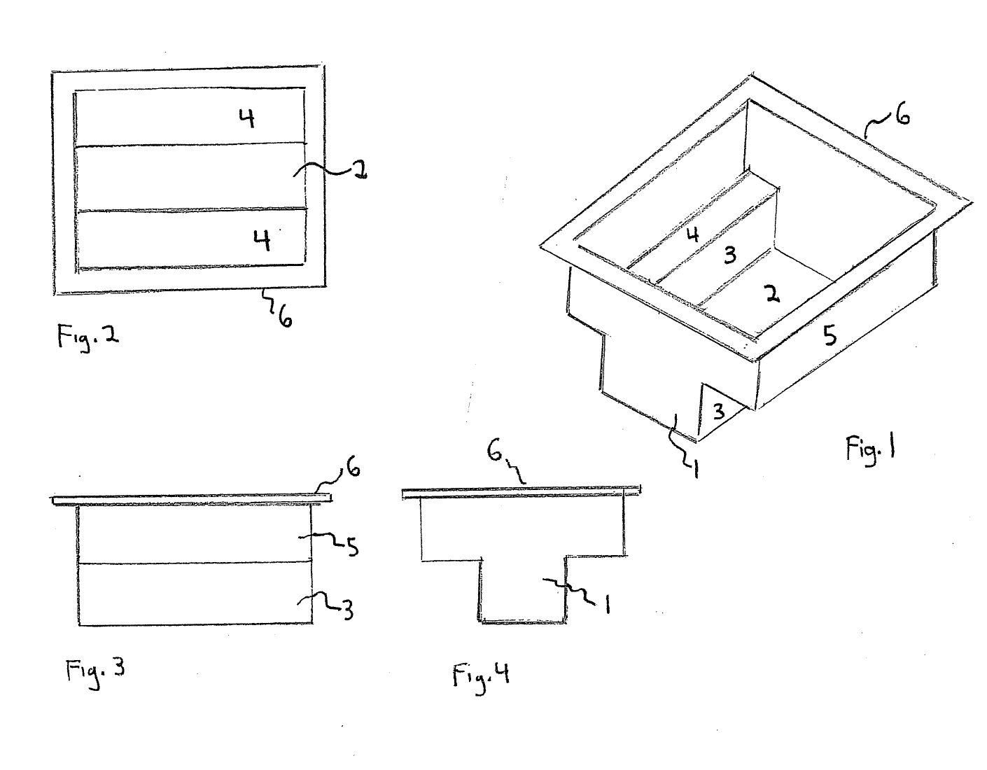

A sterile, disposable surgical tray is illustrated in FIGS. 1-4 of the drawings. The tray includes a bottom surface 2, FIGS. 1 and 2, which creates a bottom tier having two opposed, longitudinally oriented sides 3, FIGS. 1 and 3, and two opposed, widthwise oriented sides 1, FIGS. 1 and 4, where the longitudinally oriented sides 3 are substantially longer than the widthwise oriented sides 1.

A top tier sits atop the bottom tier with the top tier being composed of two opposed, longitudinally oriented sides 5, FIGS. 1 and 3, that run at least the length of the bottom tier's longitudinally oriented sides 3. The width of the top tier is larger than the width of the bottom tier. This creates an inner flat surface or upper bottom of the tray 4, FIGS. 1 and 2. The widthwise sides 1 from the bottom tier widen at the base of the top tier to enclose the upper bottom 4. There is also a flange 6, FIGS. 1-6, that sits on the two longitudinally oriented sides 5 and the two opposed widthwise oriented sides 1. The surface of flange 6 is what the lid is heat sealed to.

Sterile, disposable surgical trays are intended to hold a complete set of materials needed for any given type of surgery. The width of the bottom tier of the invention, the lower portion of side 1, is narrow enough to be grasped in a human palm of an upturned hand, with the thumb and fingers grasping the opposite sides 3. This lets a user hold the tray, and more importantly, grasp the tray so that a sufficient force is applied by the user's grip to keep the tray from moving when the lid is being peeled back.

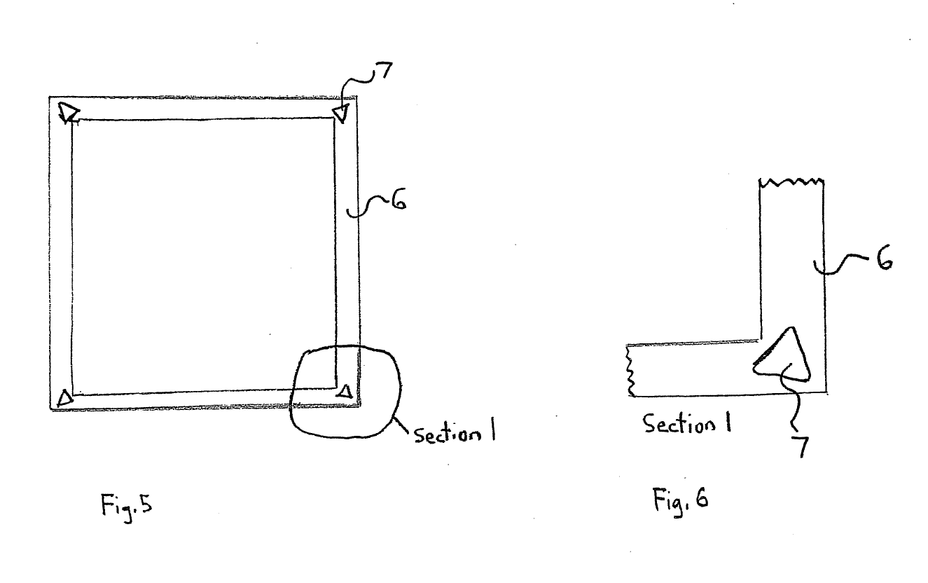

There are four protrusions 7, FIGS. 5 and 6, on the top surface of the flange 6. These protrusions enable the user to separate a corner of the lid from the flange 6 so that the lid can be pulled back. The prior art only used two protrusions 7 in diagonal corners of the flange 6, and the placement of these protrusions 7 favored right-handed users. However, the inclusion of four protrusions 7, one in each corner of the flange 6, helps both left-handed and right-handed users.

From the foregoing, what is disclosed is a novel ergonomic disposable tray for holding sterile surgical components with two tiers, where the bottom tier is of a width that can be held firmly in the user's upraised palm. The tray advantageously further has the same volume as the wide and flat bottom tray it replaces, but with the ergonomic bottom tier for easier handling.

While the preferred embodiment of this invention has been illustrated and described, it is to be understood that the invention is capable of variation and modification, and therefore the invention should not be limited to the precise details set forth, but to such changes and alterations that fall within the purview of the following claims.

What is claimed is:

1. A disposable tray for holding sterile surgical components comprising:
a bottom surface creating a bottom tier having a bottom width; and
a top tier on top of the bottom tier having a width that is larger than the bottom width; and
a flange on the top tier.

2. The tray of claim 1, wherein the bottom tier further comprises two opposed, longitudinally oriented sides and two opposed, widthwise oriented sides, wherein the longitudinally oriented sides are longer than the widthwise oriented sides.

3. The tray of claim 1, wherein the flange has four protrusions, one in each corner, to aid both right and left handed users in peeling back the lid of the tray.
